(19) United States
(12) Patent Application Publication
(10) Pub. No.: US 2024/0278031 A1
(43) Pub. Date: Aug. 22, 2024

(54) METHOD AND APPARATUS FOR PROVIDING PULSED ELECTROMAGNETIC FIELD THERAPY

(71) Applicant: Regenesis Biomedical, Inc., Scottsdale, AZ (US)

(72) Inventors: John Y. BABICO, Scottsdale, AZ (US); Brian MOWER, Scottsdale, AZ (US); Riley GILL, Scottsdale, AZ (US)

(21) Appl. No.: 18/568,493

(22) PCT Filed: Jun. 8, 2022
(86) PCT No.: PCT/US2022/032625

§ 371 Date: Dec. 8, 2023

Related U.S. Application Data
(60) Provisional application No. 63/208,944, filed on Jun. 9, 2021.

(51) Int. Cl. A61N 2/02; H01F 27/28

(52) U.S. Cl. CPC A61N 2/02; H01F 27/28

(57) ABSTRACT

A method and apparatus for providing pulsed electromagnetic field (PEMF) therapy to a patient is disclosed. In some embodiments, the PEMF therapy may be provided through an applicator that includes one or more PEMF emitter coils and a ferromagnetic field director. The ferromagnetic field director can direct a magnetic field away from an unused side of the applicator and towards the patient. In some embodiments, the applicator may include alternative therapy pads (e.g., heating pads, cooling pads, transcutaneous electrical nerve stimulation (TENS) pads, and the like) in addition to the PEMF emitter coils.

### CROSS REFERENCE TO RELATED APPLICATIONS

None

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

This disclosure relates generally to pulsed electromagnetic field (PEMF) systems, apparatuses, and methods. In particular, the disclosure relates to applicators for providing electromagnetic field therapy to one or more body regions of a patient.

### BACKGROUND

Pulsed electromagnetic fields (PEMF) have been described for treating therapeutically resistant problems of both the musculoskeletal system as well as soft tissues. PEMF typically includes the use of low-energy, time-varying magnetic fields. For example, PEMF therapy has been used to treat non-union bone fractures and delayed union bone fractures. PEMF therapy has also been used for treatment of corresponding types of body soft tissue injuries including chronic refractory tendinitis, decubitus ulcers and ligament, tendon injuries, osteoporosis, and Charcot foot. During PEMF therapy, an electromagnetic transducer coil is generally placed in the vicinity of the injury (sometimes referred to as the “target area”) such that pulsing the transducer coil will produce an applied or driving field that penetrates to the underlying tissue.

Treatment devices emitting magnetic and/or electromagnetic energy offer significant advantages over other types of electrical stimulators because magnetic and electromagnetic energy can be applied externally through clothing and wound dressings, thereby rendering such treatments completely non-invasive. Moreover, published reports of double-blind placebo-controlled clinical trials utilizing a RF transmission device (Diapulse) suggest that this ancillary treatment device significantly reduces wound healing time for chronic pressure ulcers as well as for surgical wounds. Studies using Dermagen, a magnetic device manufactured in Europe which produces a low frequency magnetic field, have demonstrated significant augmentation of healing of venous stasis ulcers. Additionally, it has been shown for groups of patients treated with electromagnetic energy, that 50% fewer patients of that treatment group develop reoccurring pressure ulcers, compared to control patients, suggesting that electromagnetic energy treatments impart some resistance to the reoccurrence of chronic wounds, such as pressure ulcers. Electromagnetic energy may also be useful as a preventative strategy. Analysis of the effects of electromagnetic energy on the treatment of pressure ulcers show that this treatment, by reducing healing time by an average of 50%, results in significant reductions in the costs associated with wound management.

### SUMMARY OF THE DISCLOSURE

In general, described herein are pulsed electromagnetic field (PEMF) apparatuses (e.g., devices and systems) including a PEMF therapy device coupled to a PEMF applicator that is configured to direct magnetic fields away from an unused side of the applicator and toward a patient's body part. In some embodiments, the PEMF applicator may include a ferromagnetic field director that may be configured to direct a magnetic field away from the unused side of the PEMF applicator and toward the patient. In some other embodiments, the PEMF applicator may include additional therapy pads. The additional therapy pads may provide alternative (heating, cooling, transcutaneous electrical nerve stimulation, or the like) therapies before, during, or after PEMF therapy.

One aspect of subject matter described herein may be implemented in a PEMF therapy applicator. The PEMF applicator may include a substrate, a ferromagnetic field director comprising a first side attached to a first surface of the substrate and a second side opposite to the first side, and an emitter coil array disposed on the second side of the ferromagnetic field director, wherein the ferromagnetic field director is configured to receive a magnetic field from the emitter coil array and direct the received magnetic field back toward the emitter coil array.

The emitter coil array may include a first PEMF emitter coil configured to emit a magnetic field toward the ferromagnetic field director and a second PEMF emitter coil configured to emit a magnetic field away from the ferromagnetic field director. In some embodiments, the first PEMF emitter coil and the second PEMF emitter coil may both be configured to spiral in the same direction. In other embodiments, the first PEMF emitter coil and the second PEMF emitter coil may both be configured to spiral in the opposite direction. In still other embodiments, the first PEMF emitter coil may be configured to emit a magnetic field having a north-south orientation toward the ferromagnetic field director and the second PEMF emitter coil may be configured to emit a magnetic field having a north-south orientation away from the ferromagnetic field director.

In some embodiments, the first PEMF emitter coil may be disposed adjacent to the second PEMF emitter coil. In some other embodiments, the second PEMF emitter coil may be coupled in series with the first PEMF emitter coil. Furthermore, current flow in the first and second PEMF emitter coils may be in opposite directions with respect to a PEMF emitter coil spiral direction.

In some embodiments, the ferromagnetic field director may be further configured to direct the magnetic field from the first PEMF emitter coil to the second PEMF emitter coil. In some other embodiments, the ferromagnetic field director may include a plurality of individual ferromagnetic field directors, where each individual ferromagnetic field director is disposed under two PEMF emitter coils of the emitter coil array.

In some embodiments, the ferromagnetic field director may include at least one metal selected from the group consisting of cobalt, iron, nickel, and gadolinium. In some embodiments, the substrate may be a printed circuit board. The PEMF therapy applicator may include an adhesive configured to temporarily affix the PEMF therapy applicator to a selected body part. The PEMF therapy applicator may also include a visual cue configured to guide positioning of a body part with respect to the emitter coil array.

Another innovative aspect of subject matter described herein may be implemented as a pulsed electromagnetic field (PEMF) system including a PEMF therapy device configured to control a generation of a PEMF signal and a PEMF applicator coupled to the PEMF therapy device and configured to deliver the PEMF signal to a patient. The PEMF applicator may include a substrate, a ferromagnetic field director comprising a first side attached to a first surface of the substrate and a second side opposite to the first side, and an emitter coil array disposed on the second side of the ferromagnetic field director, where the ferromagnetic field director is configured to receive a magnetic field from the emitter coil array and direct the received magnetic field back toward the emitter coil array.

In some embodiments, the emitter coil array may include a first PEMF emitter coil configured to emit a magnetic field toward the ferromagnetic field director and a second PEMF emitter coil configured to emit a magnetic field away from the ferromagnetic field director. Furthermore, the first PEMF emitter coil and the second PEMF emitter coil may both be configured to spiral in the same direction. Still further, the first PEMF emitter coil and the second PEMF emitter coil may both be configured to spiral in the opposite directions.

In some embodiments, the first PEMF emitter coil may be configured to emit a magnetic field having a north-south orientation toward the ferromagnetic field director and the second PEMF emitter coil may be configured to emit a magnetic field having a north-south orientation away from the ferromagnetic field director. Furthermore, the first PEMF emitter coil may be disposed adjacent to the second PEMF emitter coil. In some embodiments, the second PEMF emitter coil may be coupled in series with the first PEMF emitter coil. In some other embodiments, current flow in the first and second PEMF emitter coils are in opposite directions with respect to a PEMF emitter coil spiral direction. In still other embodiments, the ferromagnetic field director may be further configured to direct the magnetic field from the first PEMF emitter coil to the second PEMF emitter coil.

In some embodiments, the ferromagnetic field director may include a plurality of individual ferromagnetic field directors, wherein each individual ferromagnetic field director is disposed under two PEMF emitter coils of the emitter coil array. In some other embodiments, the ferromagnetic field director may include at least one metal selected from the group consisting of cobalt, iron, nickel, and gadolinium.

In some embodiments, the substrate may be a printed circuit board. In some other embodiments, the PEMF applicator may include an adhesive configured to temporarily affix the PEMF applicator to a selected body part. In some other embodiments, the PEMF applicator further comprises a visual cue configured to guide positioning of a body part with respect to the emitter coil array.

Yet another innovative aspect of subject matter described herein may be implemented as a combination therapy applicator including a substrate, one or more pulsed electromagnetic (PEMF) emitter coils disposed on the substrate and configured to provide a first therapy to a patient, wherein the first therapy is a pulsed electromagnetic field therapy and one or more therapy pads disposed on the substrate configured to provide a second therapy to the patient, wherein the second therapy is different than the first therapy.

In some embodiments, the one or more therapy pads may be heating pads, cooling pads, transcutaneous electrical nerve stimulation (TENS) pads, or a combination thereof. In some other embodiments, the combination therapy applicator may include a ferrite sheet coupled to the substrate and configured to receive a magnetic field from the one or more PEMF emitter coils and direct the received magnetic field back toward the one or more PEMF emitter coils. The ferrite sheet may be disposed under the one or more PEMF emitter coils and is absent from under the one or more therapy pads. In some embodiments, the substrate may be shaped to conform to a particular body part. The particular body part may be a foot, a hand, a wrist, an arm, a leg, a waist, a back, or a combination thereof.

Yet another innovative aspect of subject matter described herein may be implemented as a method for providing pulsed electromagnetic field (PEMF) treatment. The method may include positioning a combination therapy applicator proximate to a selected body part of a patient, where the combination therapy applicator may include one or more PEMF emitter coils disposed on a substrate and configured to provide a first therapy to the patient, wherein the first therapy is a pulsed electromagnetic field therapy and one or more therapy pads disposed on the substrate configured to provide a second therapy to the patient, wherein the second therapy is different than the first therapy. The method may also include providing, to the patient, the first therapy and providing, to the patient, the second therapy.

In some embodiments, the first therapy may be provided for a first time period and the second therapy is provided for a second time period. Furthermore, the first time period may be prior to the second time period. In some other embodiments, the first time period may overly the second time period. In still other embodiments, the second therapy may include a heat therapy, a cooling therapy, a transcutaneous electrical nerve stimulation (TENS) therapy, or a combination thereof.

### BRIEF DESCRIPTION OF THE DRAWINGS

Novel features of embodiments described herein are set forth with particularity in the appended claims. A better understanding of the features and advantages of the embodiments may be obtained by reference to the following detailed description that sets forth illustrative embodiments and the accompanying drawings.

FIG. 1 is a diagram of a pulsed electromagnetic field (PEMF) system, according to some embodiments.

FIG. 2 shows an example emitter coil array, in accordance with some embodiments.

FIG. 3 shows another example emitter coil array, in accordance with some embodiments.

FIG. 4 shows a block diagram of another example emitter coil array 40, in accordance with some embodiments.

FIG. 5 is an example PEMF applicator, in accordance with some embodiments.

FIG. 6 is another example PEMF applicator, in accordance with some embodiments.

FIG. 7 shows an example combination applicator, in accordance with some embodiments.

FIG. 8 shows another example combination applicator, in accordance with some embodiments.

FIG. 9 shows another example combination applicator, in accordance with some embodiments.

FIG. 10 shows another example combination applicator, in accordance with some embodiments.

FIG. 11 shows another example combination applicator, in accordance with some embodiments.

FIG. 12 is a flowchart depicting an example of one method for providing therapy to a patient with a combination applicator.

FIG. 13 shows a block diagram of a PEMF therapy device that may be one example of the PEMF therapy device of FIG. 1.

### DETAILED DESCRIPTION

Pulsed electromagnetic fields (PEMF) have been used to treat patients by, for example, providing low-energy, time-varying magnetic fields to targeted body parts. For example, PEMF therapy has been used to treat non-union bone fractures and delayed union bone fractures. PEMF therapy has also been used for treatment of corresponding types of body soft tissue injuries including chronic refractory tendinitis, decubitus ulcers and ligament, tendon injuries, osteoporosis, and Charcot foot. During PEMF therapy, an electromagnetic transducer coil (sometimes referred to as an emitter coil) is generally placed in the vicinity of the injury (sometimes referred to as a target area) such that pulsing the transducer coil will produce an applied or driving field that penetrates to the underlying tissue.

PEMF therapy treatments may be delivered through applicators that include a PEMF emitter coil. However, a single PEMF emitter coil may not provide a uniform magnetic field, particularly over a larger area, such as a person's foot, hand, arm, or the like. A more uniform magnetic field may be provided by applicators that include a plurality of PEMF emitter coils. Furthermore, the PEMF emitter coils may be arranged to direct a magnetic field substantially in one direction with respect to the applicator. In this manner, the magnetic field may be concentrated toward the patient and not directed toward an empty region.

In some embodiments, the applicator may include one or more therapy pads in addition to the PEMF emitter coils. In this manner, the applicator may deliver other treatment therapies to the patient. For example, the therapy pads may include heating, cooling, and/or transcutaneous electrical nerve stimulation (TENS) pads. Thus, alternative treatment may be provided to the patient through the therapy pads before, during, or after PEMF treatment is provided.

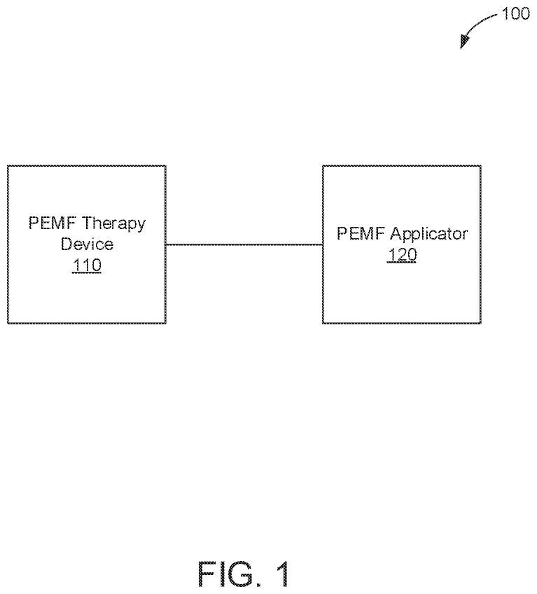

FIG. 1 is a diagram of a PEMF system 100, according to some embodiments. The PEMF system 100 may include a PEMF therapy device 110 coupled to a PEMF applicator 120.

The PEMF therapy device 110 may be used to deliver one or more high-power, pulsed electromagnetic fields to a patient through one or more PEMF applicators, such as the PEMF applicator 120. Although only one PEMF applicator 120 is shown, in other embodiments, the PEMF system 100 may include any feasible number of PEMF applicators 120. The high-power pulsed electromagnetic fields may provide a therapeutic effect to the patient in a non-invasive manner. In some examples, the high-power pulsed electromagnetic fields may upregulate cytokines, collagen, alpha SMA, FGF and other markers associated with wound healing. In still other examples, the high-power pulsed electromagnetic fields may treat inflammation and tissue remodeling associated with a predicted or pending diabetic foot ulcers and/or pressure ulcers.

The PEMF applicator 120 may be configured to emit a directed magnetic field. For example, the PEMF applicator 120 may include PEMF emitters that may be configured to emit a magnetic field substantially in a first direction while not emitting a magnetic field in an opposite direction. In another example, the PEMF applicator 120 may be a combination applicator that includes a first set of emitters and a second set of emitters. The first set of emitters may include
PEMF emitters, and the second set of emitters may include heating pads, cooling pads, transcutaneous electrical nerve stimulation (TENS) pads, or any other feasible pad or emitter.

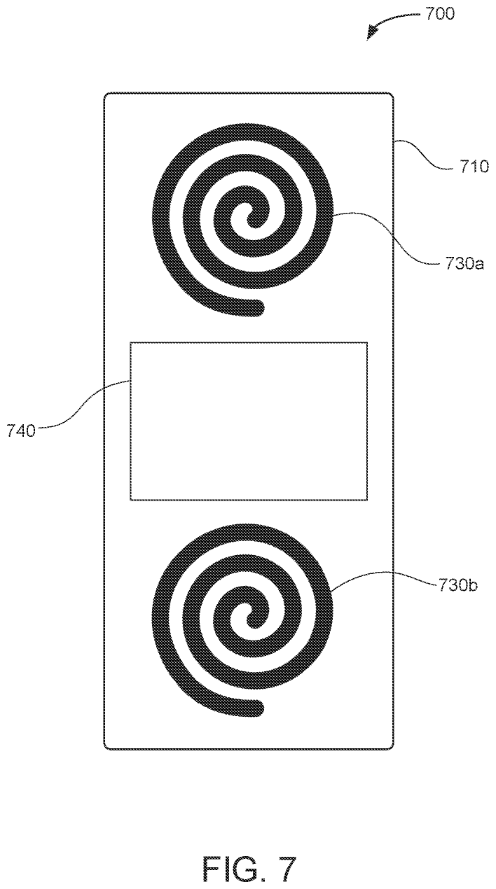

The PEMF therapy device 110 may control, at least in part, electromagnetic fields generated by the PEMF applicator 120. For example, the PEMF therapy device 110 may generate and provide a high-power pulsed PEMF signals to the PEMF applicator 120. The PEMF applicator 120 can use PEMF signals to generate and radiate therapeutic electromagnetic fields. In another example, the PEMF applicator 120 may include circuits that can receive the voltage and/or control signals from the PEMF therapy device 110 and, in turn, locally generate the therapeutic electromagnetic fields. If the PEMF applicator 120 includes alternative therapy pads (heating, cooling, TENS, and the like), then the PEMF therapy device 110 may also cause the alternative therapy pads to provide the associated alternative therapy. For example, the PEMF therapy device 110 may include circuits, drivers, and the like to cause the alternative therapy pads to provide heating, cooling, TENS therapy, or the like. Alternative therapy pads are described in more detail with respect to FIGS. 7-12.

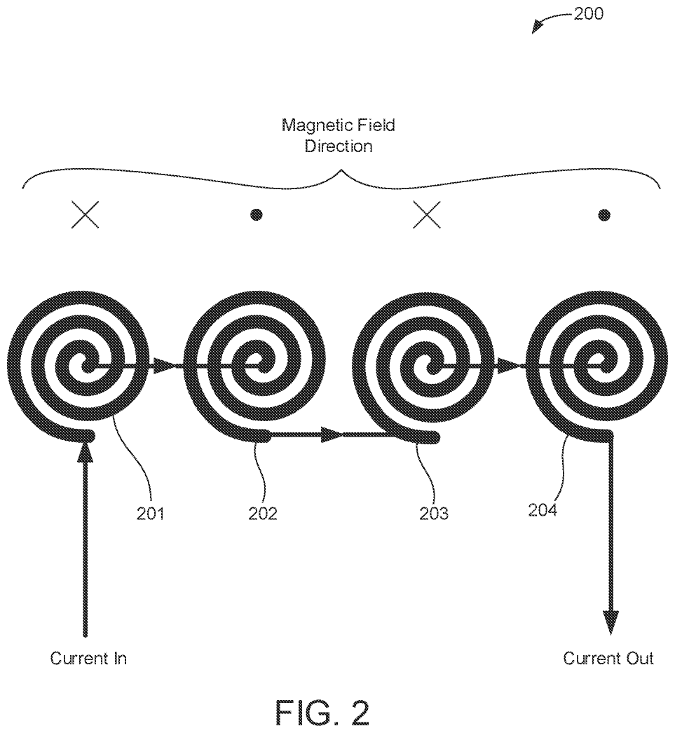

FIG. 2 shows an example emitter coil array 200, in accordance with some embodiments. The emitter coil array 200, which may also be referred to as a PEMF emitter coil array, may be included with any feasible PEMF applicator, such as the PEMF applicator 120 of FIG. 1. The emitter coil array 200 may include four PEMF emitter coils 201-204, however in other embodiments, the emitter coil array 200 may include any feasible number of PEMF emitter coils.

The PEMF emitter coils 201-204 may be configured to radiate magnetic fields in alternating directions. For example, PEMF emitter coil 201 may be configured such that the magnetic field radiates into the page (denoted by the “X” above PEMF emitter coil 201). In contrast, PEMF emitter coil 202 may be configured to radiate a magnetic field out of the page (denoted by the dot above PEMF emitter coil 202). In a similar manner, PEMF emitter coil 203 may radiate a magnetic field into the page and PEMF emitter coil 204 may radiate a field out of the page. In some cases, the magnetic field direction may be from a magnetic north pole of a PEMF emitter coil toward a magnetic south pole of the PEMF emitter coil. For example, the magnetic field of PEMF emitter coil may proceed from a north pole to a south pole of the PEMF emitter coil into the page. In some other variations, the magnetic field direction may be from a magnetic south pole to a magnetic north pole.

As shown in the emitter coil array 200, the direction of current within each of the PEMF emitter coils 201-204 can alternate from outer conductor to inner connector (for example, as shown for PEMF emitter coils 201 and 203) to inner conductor to outer conductor (for example, as shown for PEMF emitter coils 202 and 204). Alternating the direction of current with respect to adjacent (e.g., nearest) emitter coils may cause the direction of the magnetic field to alternate as shown due, for example, to the right hand rule of magnetic forces.

As depicted, the PEMF emitter coils 201-204 may each spiral clockwise from outer conductor to inner conductor. In order to achieve alternating magnetic field directions, the PEMF emitter coils 201 and 203 may be arranged so that current enters their outer conductors and the PEMF emitter coils 202 and 204 may be arranged so that current enters their inner conductors.

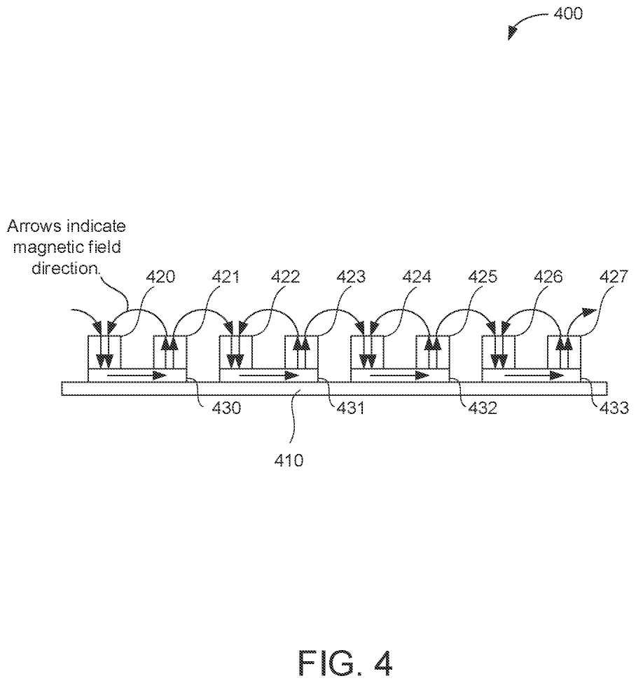

In some embodiments, the alternating direction of magnetic fields from adjacent PEMF emitter coils may enable a directional increase of the magnetic fields from the PEMF emitter coils 201-204 with respect to the emitter coil array 200. In some cases, the alternating magnetic fields may enable a doubling of a magnetic field strength. The increase in magnetic field strength is described in more detail below with respect to FIG. 4.

In some embodiments, the PEMF emitter coils 201-204 may be substantially similar. That is, each of the PEMF emitter coils 201-204 may be composed of similar materials and may have a similar construction. For example, the PEMF emitter coils 201-204 may each have a similar number of turns, and spiral in a similar direction (in this example, clockwise, from outer conductor to inner conductor). In other embodiments, the PEMF emitter coils 201-204 may each have a different construction. That is, some of the PEMF emitter coils 210-204 may spiral in a clockwise direction while some of the other coils may have a counter-clockwise direction. Furthermore, the PEMF emitter coils may have varying number of turns.

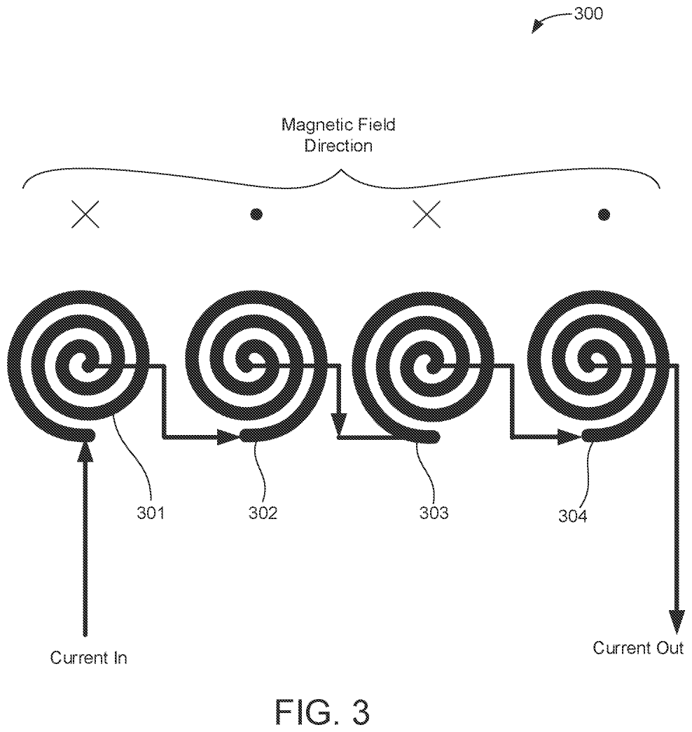

FIG. 3 shows another example emitter coil array 300, in accordance with some embodiments. The emitter coil array 300, which also may be referred to as a PEMF emitter coil array, may be included with any feasible PEMF applicator, such as the PEMF applicator 120 of FIG. 1. The emitter coil array 300 may include four PEMF emitter coils 301-304, however in other embodiments, the emitter coil array 300 may include any feasible number of PEMF emitter coils.

Similar to the emitter coil array 200 of FIG. 2, the PEMF emitter coils 301-304 of the emitter coil array 300 may be configured to radiate magnetic fields in alternating directions with respect to each adjacent PEMF emitter coil. Thus, in the example of FIG. 3, the direction of the magnetic field for PEMF emitter coils 301 and 303 may be into the page and the direction of the magnetic fields for PEMF emitter coils 302 and 304 may be out of the page. As described above, the alternating direction of magnetic fields from the adjacent emitter coils may enable a directional increase of the magnetic field with respect to the emitter coil array 300.

In contrast to the PEMF emitter coils 201-204, the PEMF emitter coils 301-304 may not be substantially similar. For example, PEMF emitter coils 301-304 do not all spiral clockwise from outer conductor to inner conductor. As shown, PEMF emitter coils 301 and 303 may spiral clockwise from outer conductor to inner conductor and PEMF emitter coils 302 and 304 may spiral counter-clockwise from outer conductor to inner conductor. In order to achieve alternating magnetic field directions, the PEMF emitter coils 301-304 may be arranged so that current enters the outer conductor of the coils. In some embodiments, changing the current direction (e.g., clockwise versus counter-clockwise) for PEMF emitters with different spiral directions may cause an alternating direction of magnetic fields due to the right-hand rule of magnetic forces. In this manner, the alternating spirals of the coils 301-304 may produce an alternating direction of magnetic fields similar to those described in FIG. 2.

FIG. 4 shows a block diagram of another example emitter coil array 400, in accordance with some embodiments. The emitter coil array 400, shown here in a side view, may include a substrate 410, PEMF emitter coils 420-427, and ferromagnetic field directors 430-433. In some embodiments, the arrangement of the PEMF emitter coils 420-427 with respect to the ferromagnetic field directors 430-433 may direct magnetic fields from the PEMF emitter coils 420-427 in a particular direction.

In one example, the substrate 410 may be a printed circuit board. In other embodiments, the substrate 410 may be any feasible rigid, semi-rigid, or flexible material. The ferromagnetic field directors 430-433 may disposed on a first surface of the substrate 410. For example, a first surface of the ferromagnetic field directors 430-433 may be disposed on (e.g., coupled to, and/or in contract with) the first surface of the substrate 410. The ferromagnetic field directors 430-433 may be formed from and/or include any feasible material, particularly material with a high relative permeability. Relative permeability may refer to a materials ability to attract and conduct magnetic lines of flux (associated with, for example, a magnetic field). Example materials for ferromagnetic field directors 430-433 may include cobalt, iron, nickel, gadolinium, and the like. In some cases, the ferromagnetic field directors 430-433 may be formed from an alloy that includes cobalt, iron, nickel, gadolinium, or the like. In some embodiments, the ferromagnetic field directors 430-433 can conduct or direct a magnetic field (e.g., magnetic flux lines) from one PEMF emitter coil to another.

The PEMF emitter coils 420-427 may each include a first surface and a second surface opposite to the first surface. Furthermore, the first surface of each of the PEMF emitter coils 420-427 may be disposed on a second surface (e.g., the surface not in contact with the substrate 410) of the ferromagnetic field directors 430-433. Thus, the ferromagnetic field directors 430-434 may be disposed between the PEMF emitter coils 420-427 and the substrate 410.

As described with respect to FIGS. 2 and 3, the PEMF emitter coils 420-427 may be configured so that the direction of the magnetic fields emitted by each PEMF emitter coil alternates for adjacent PEMF emitter coils. In some examples, the direction of the magnetic fields may be determined, at least in part, by an electrical current direction and/or physical characteristics of the PEMF emitter coils, including, but not limited to a spiral direction. The direction of the magnetic fields is illustrated in FIG. 4 with arrows. Therefore, the magnetic fields for the PEMF emitter coils 420, 422, 424, and 426 may be directed toward the substrate 410 and the magnetic fields for the PEMF emitter coils 421, 423, 425, and 427 may be directed away from the substrate 410.

As shown, the ferromagnetic field directors 430-434 may direct a magnetic field from a first PEMF emitter coil to a second PEMF emitter coil. For example, the first surface of the PEMF emitter coils 420 and 421 may be disposed on the second surface of ferromagnetic field director 430. Thus, a magnetic field from the first surface of the PEMF emitter coil 420 may be directed by the ferromagnetic field director 430 to the first surface of the PEMF emitter coil 421. The PEMF emitter coils 422-427 and the ferromagnetic field directors 431-433 may be arranged in similar manner.

In this manner, the magnetic fields that are radiated from the first surfaces of the PEMF emitter coils 420, 422, 424, and 426 may be directed toward the first surfaces of the PEMF emitter coils 421, 423, 425, and 427. Since the magnetic fields from PEMF emitter coils 420, 422, 424, and 426 are not radiated and lost into the surrounding area (e.g., an area in and around the substrate 410), the strength of the magnetic fields on the second surface of the PEMF emitter coils 420-427 may be increased. In some embodiments, the strength of the magnetic field near the second surface of the PEMF emitter coils 420-427 may be twice the strength compared to implementations of an emitter coil array that lack the ferromagnetic field directors 430-433.

Persons having skill in the art will recognize that a magnetic field uniformity may be based, at least in part, on a spacing and number of the PEMF emitter coils 420-427. For example, the more spread out and separated that the PEMF emitter coils 420-427 are from each other, the less uniform the resulting magnetic field may be. On the other hand, the closer at the PEMF emitter coils 420-427 are positioned with respect to each other, then the more uniform (e.g., less magnetic field ripple) the magnetic field may be. Thus, an applicator having more PEMF emitter coils may provide a more uniform magnetic field than a PEMF applicator with fewer or more spread out PEMF emitter coils.

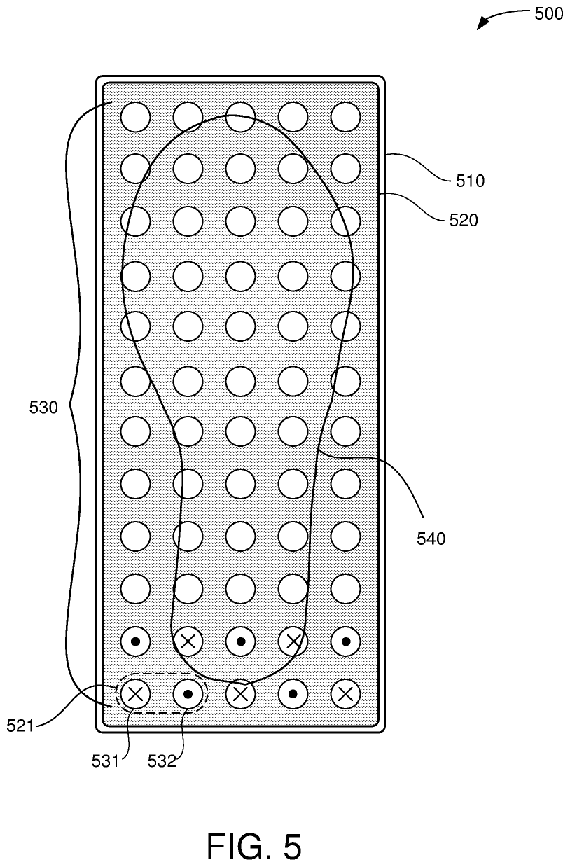

FIG. 5 is an example PEMF applicator 500, in accordance with some embodiments. The PEMF applicator 500 may be particularly adapted for use with a patient's foot, however other PEMF applicators may be adapted to provide therapeutic PEMF therapy to other portions or regions of a patient's body. For example, other example PEMF applicators may be adapted for use on a patient's hand, arm, leg, or the like. The PEMF applicator 500 may include a substrate 510, a ferrite sheet 520, and an emitter coil array 530.

The substrate 510 may be flexible and may easily conform to any feasible body part of the patient. The substrate 510 may be feasible material such as any durable, rigid, flexible, or partially flexible material including a natural or synthetic fabric, rubber, synthetic rubber, nylon, fiberglass, polymer, or the like. In some embodiments, the substrate 510 may be a flexible, rigid, or semi-rigid printed circuit board (PCB). A first surface or side of the ferrite sheet 520 may be bonded or otherwise attached to one side of the substrate 510.

The emitter coil array 530 may be disposed on a second surface or side of the ferrite sheet 520. Each PEMF emitter coil of the emitter coil array 530 may include a PEMF emitter coil such as one or more of the PEMF emitter coils 201-204 of FIG. 2, the PEMF emitter coils 301-304 of FIG. 3, or the PEMF emitter coils 420-427 of FIG. 4. The PEMF emitter coils included within the emitter coil array 530 may be configured such that adjacent PEMF emitter coils emit/radiate magnetic fields in opposite directions. For example, a PEMF emitter coil 531 can generate a magnetic field into the sheet (as denoted by the “X”) and a PEMF emitter coil 532 can generate a magnetic field out of the sheet (as denoted by the dot).

The ferrite sheet 520 may direct magnetic fields from one PEMF emitter coil to another in the emitter coil array 530 similar to as described with respect to the ferromagnetic field directors 430-434 of FIG. 4. The ferrite sheet 520 may be formed and/or include any suitable ferromagnetic material. As shown, the PEMF applicator 500 may include a single ferrite sheet 520. In other embodiments, the PEMF applicator 500 may include a plurality of ferrite sheets 520, for example, where each of the plurality of ferrite sheets are disposed beneath two PEMF emitter coils of the emitter coil array 530. One example of an alternative ferrite sheet 521 is shown in dashed lines in FIG. 5.

In some embodiments, the arrangement of the PEMF emitter coils within the emitter coil array 530 upon the ferrite sheet 520 may direct magnetic fields away from the substrate 510. For example, the strength of the magnetic field above the emitter coil array 530 (e.g., above the second surface or side of the ferrite sheet 520) may be twice the strength of a magnetic field for a similar emitter coil array 530 not disposed on the ferrite sheet 520. As discussed with respect to FIG. 4, the increase in magnetic field strength may be due to the interactions between the ferrite sheet 520 and the magnetic fields from the emitter coil array 530. In some examples, the ferrite sheet 520 may be optional. Thus, in some embodiments, the ferrite sheet 520 may be omitted from the PEMF applicator 500. Without the ferrite sheet 520, magnetic field strength may not be increased as described above.

The PEMF applicator 500 may include a visual cue 540. The visual cue 540 may guide the patient to position his/her body part with respect to the PEMF applicator 500. In this manner, the patient's body part may be positioned with respect to the PEMF applicator 500 to receive an optimal magnetic field treatment. Thus, the visual cue 540 may guide positioning of the patient's body part with respect to the emitter coil array 530. In some embodiments, the visual cue 540 may be an outline of the patient's body part to be placed in contact with the PEMF applicator 500.

In some variations, the PEMF applicator 500 may be temporarily adhered and/or attached to the patient. For example, a surface of the PEMF applicator 500 may include a dressing, adhesive, or the like that allows the PEMF applicator 500 to become and/or remain attached to the body part to receive the PEMF treatment. In another variation, the PEMF applicator 500 may be affixed to the patient by means of a garment. For example, a tubular sleeve, sock or other garment or material may be used to hold the PEMF applicator 500 in proper position with respect to a patient's body part to receive PEMF therapy. Temporarily attaching the PEMF applicator 5500 to the patient may enable a more consistent and/or uniform PEMF treatment to be provided.

In some variations, the PEMF applicator 500 may be disposable. For example, the PEMF applicator 500 may be low-cost applicator that includes an adhesive to affix the applicator to the patient, as described above. After use, the PEMF applicator 500 may be discarded. Thus, a clean and/or sterile PEMF applicator 500 may be affixed to each patient for treatment, and then discarded.

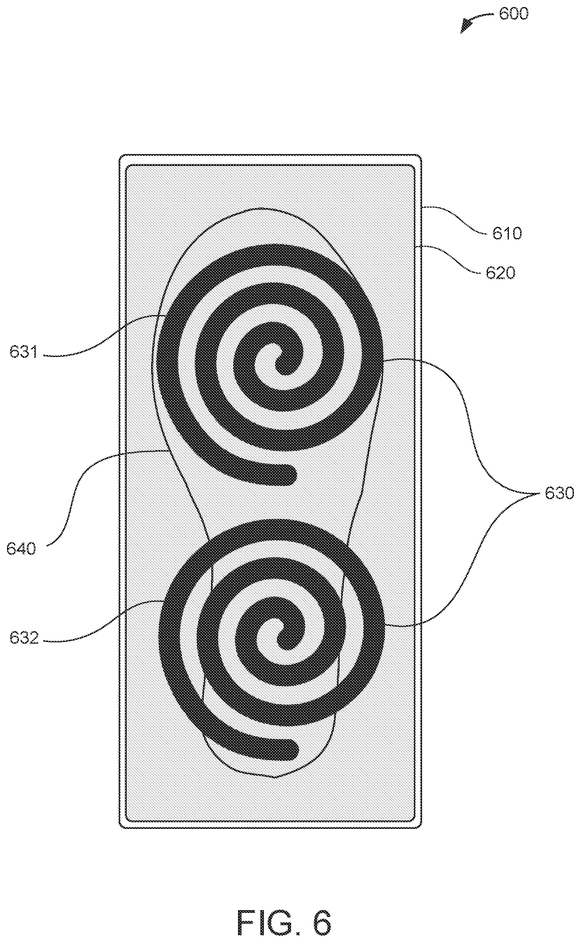

FIG. 6 is another example PEMF applicator 600, in accordance with some embodiments. The PEMF applicator 600 may be particularly adapted for use with a patient's foot, however other embodiments may be adapted to provide therapeutic PEMF therapy to other portions or regions of a patient's body. The PEMF applicator 600 may be a variation of the PEMF applicator 500 of FIG. 5 and include a substrate 610, a ferrite sheet 620, and an emitter coil array 630.

The substrate 610 may be rigid, flexible, or partially flexible and able to easily conform to contours of a patient's foot. In some examples, the substrate 610 may be formed from and/or include any feasible material including a natural or synthetic fabric, rubber, synthetic rubber, nylon, fiberglass, polymer, a flexible printed circuit board, or the like. The ferrite sheet 620 may be any feasible material to direct magnetic field lines from the emitter coil array 630. For example, the ferrite sheet 620 may be formed from and/or include any suitable ferromagnetic material. In some examples, the ferrite sheet 620 may be optional. Thus, in some embodiments, the ferrite sheet 620 may be omitted from the PEMF applicator 600. Without the ferrite sheet 620, the magnetic field strength may not be increased, for example, as described above with respect to FIG. 4.

The emitter coil array 630 may include a first PEMF emitter coil 631 and a second PEMF emitter coil 632. In some examples, using the first and second PEMF emitter coils 631 and 632 may provide a more uniform magnetic field to the patient's body part, compared to a single PEMF emitter coil. The first and second PEMF emitter coils 631 and 632 may be any feasible emitter coil. Although depicted as similar coils, in other embodiments, the first and second PEMF emitter coils 631 and 632 may be different. For example, the first PEMF emitter coil 631 may spiral clockwise from outer conductor to inner conductor and the second PEMF emitter coil 632 may spiral counter-clockwise from outer conductor to inner conductor. In still other embodiments, the first and second PEMF emitter coils 631 and 632 may have different number of spirals and/or may occupy a different amount of area.

The PEMF applicator 600 may include a visual cue 640. The visual cue 640 may guide the patient to position his/her body part with respect to the PEMF applicator 600. In this manner, the patient's body part may be positioned with respect to the PEMF applicator 600 receive an optimal magnetic field treatment. Thus, the visual cue 640 may guide positioning of the patient's body part with respect to the emitter coil array 630. In some embodiments, the visual cue 640 may be an outline of the patient's body part to be placed in contact with the PEMF applicator 600.

In some cases, a combination applicator may include one or more PEMF emitter coils and one or more additional treatment pads. The additional treatment pads may provide other modes of treatment (other than PEMF treatment) to the patient. For example, the additional treatment pads may include cooling pads, heating pads, or a transcutaneous electrical nerve stimulation (TENS) pads to provide cooling therapy, heating therapy, or TENS therapy. Thus, combination applicators may provide a combination treatment to the patient using a convenient, single applicator.

FIG. 7 shows an example combination applicator 700, in accordance with some embodiments. The combination applicator 700 may include a substrate 710, PEMF emitter coils 730a-730b (collectively referred to as PEMF emitter coils 730), and a treatment pad 740. The combination applicator 700 may be configured to deliver a combination of electromagnetic field therapy, such as PEMF therapy, and an alternate (e.g., non-PEMF) therapy to the patient without requiring the patient to exchange or change applicators. The combination applicator 700 may be coupled to a control unit (not shown for simplicity). In some embodiments, the PEMF therapy device 110 of FIG. 1 may be configured to function as the control unit for the combination applicator 700. Thus, the PEMF therapy device 110 may include circuits to control and/or generate the electromagnetic fields for the PEMF emitter coils 730 and also circuits to control the treatment pad 740.

The substrate 710 may be any suitable material that can support the PEMF emitter coils 730 and the treatment pad 740. In some examples, the substrate 710 may be a flexible or rigid PCB, a rubber mat, a polymer, a natural or synthetic fabric, or the like. In some variations, a ferrite sheet (not shown) may be attached to the substrate 710 to direct magnetic fields from the PEMF emitter coils 730. In some other embodiments, the substrate 710 and the ferrite sheet may be integrated into a single unit. In some variations, the ferrite sheet may be disposed under the PEMF emitter coils 730 and absent from under the treatment pad 740. Although shown as a rectangle, in other variations, the substrate 710 may be any feasible shape. In some variations, the substrate 710 may be shaped to conform to a particular part of the patient's body.

The PEMF emitter coils 730 may be any feasible PEMF emitter coils, including the PEMF emitter coils 201-204 of FIG. 2, the PEMF emitter coils 301-304 of FIG. 3, the PEMF emitter coils 420-427 of FIG. 4, the PEMF emitter coils 531, 532 of FIG. 5, the PEMF emitter coils 631-632 of FIG. 6, or any other feasible PEMF emitter coils. Although only two PEMF emitter coils 730 are shown, in other embodiments, the combination applicator 700 may include any feasible number of PEMF emitter coils. Positions of the PEMF emitter coils 730 and the treatment pad 740 with respect to the substrate 710 in FIG. 7 is meant to be illustrative rather than limiting.

The treatment pad 740 may be any feasible device that can deliver a treatment, other than a PEMF treatment, to the patient. Although only one treatment pad 740 is shown here, in other embodiments, the combination applicator 700 may include any feasible number of treatment pads. In a first example, the treatment pad 740 may be a heating pad. Thus, the control unit may control the delivery of a pulsed electromagnetic field therapy through the PEMF emitter coils 730 and control the generation of heat through the treatment pad 740.

In another example, the treatment pad 740 may be a cooling pad. Thus, the control unit may control the delivery of pulsed electromagnetic field therapy through the PEMF emitter coils 730 and also control cooling provided by the treatment pad 740. In yet another example, the treatment pad 740 may be TENS pad. Thus, the control unit may control the delivery of the pulsed electromagnetic field therapy through the PEMF emitter coils 730 and a TENS treatment through the treatment pad 740.

Similar to the PEMF applicator 500 of FIG. 5, the combination applicator 700 may include a dressing, adhesive or the like to affix the applicator to the patient. In some variations, the combination applicator 700 may be held in place on the patient with a tubular sleeve, sock, or other garment or material. Furthermore, in some variations, the combination applicator 700 may be disposable.

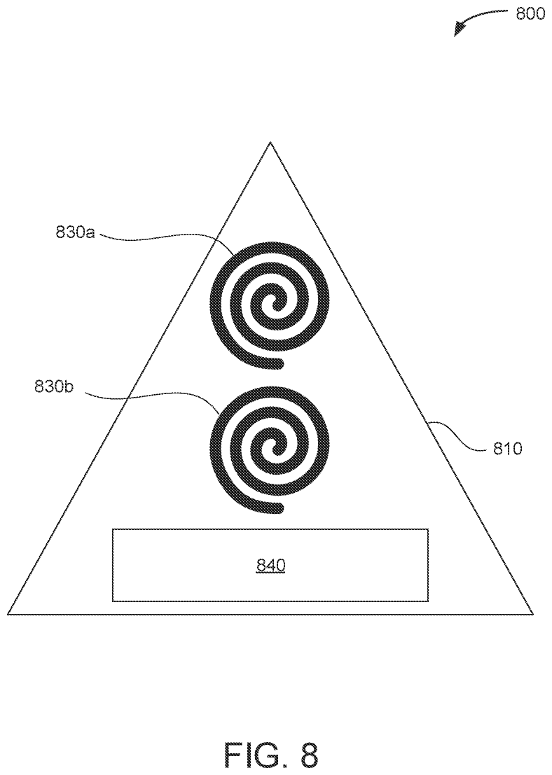

FIG. 8 shows another example combination applicator 800, in accordance with some embodiments. The combination applicator 800 may include a substrate 810, PEMF emitter coils 830a-830b (collectively referred to as PEMF emitter coils 830), and a treatment pad 840. The PEMF emitter coils 830 and the treatment pad 840 may be similar to the PEMF emitter coils 730 and treatment pad 740 of FIG. 7, respectively. The substrate 810 may formed from any feasible material. As shown, the shape of the substrate 810 may be triangular, however in other embodiments, the substrate 810 may be any feasible shape. Positions of the PEMF emitter coils 830 and the treatment pad 840 with respect to the substrate 810 in FIG. 8 is meant to be illustrative rather than limiting.

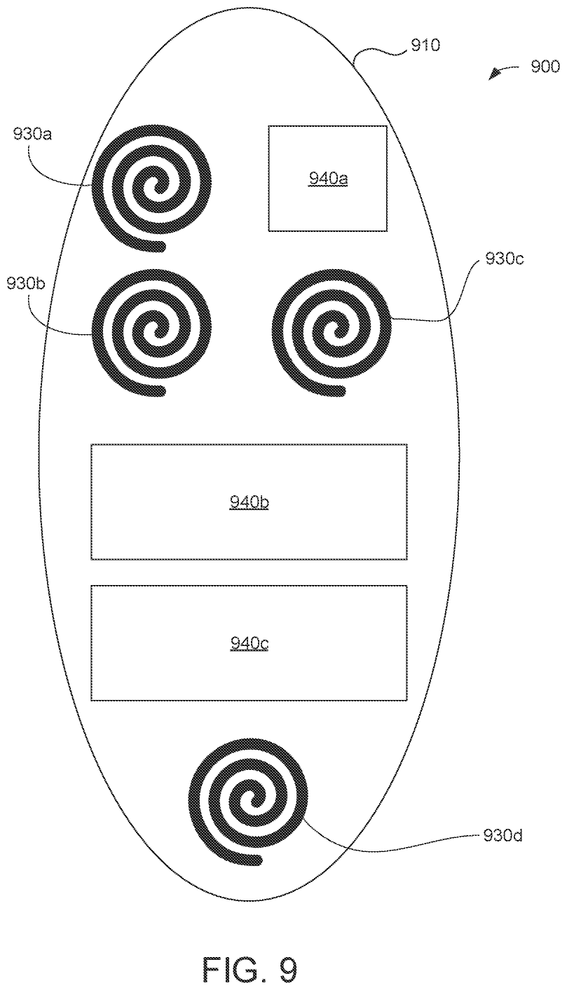

FIG. 9 shows another example combination applicator 900, in accordance with some embodiments. The combination applicator 900 may include a substrate 910, PEMF emitter coils 930a-930d (collectively referred to as PEMF emitter coils 930), and treatment pads 940a-940c (collectively referred to as treatment pads 940). The PEMF emitter coils 930 and the treatment pads 940 may be similar to the PEMF emitter coils 730 and treatment pad 740 of FIG. 7, respectively. Although shown as an oval, in other variations, the substrate 910 may be any feasible shape. Although the example combination applicator 900 depicts four PEMF emitter coils and three treatment pads, in other embodiments, the combination applicator 900 may include any feasible number of PEMF emitter coils and treatment pads. Positions of the PEMF emitter coils 930 and the treatment pads 940 with respect to the substrate 910 in FIG. 9 is meant to be illustrative rather than limiting.

In some embodiments, the treatment pads 940 may all be the same type of treatment pad. For example, the treatment pad 940a, the treatment pad 940b, and the treatment pad 940c may all be heating pads, cooling pads, TENS pads, or the like. In another embodiment, some, or all of the treatment pads 940 may be different. For example, the treatment pad 940a may be a heating pad, the treatment pad 940b may be a cooling pad and the treatment pad 940c may be a TENS pad. In other embodiments, the treatment pads 940 may include any feasible mix or types of treatment pads.

Since the combination applicator 900 may include a variety of treatment pads, several different therapies may be delivered to the patient. Furthermore, the different therapies may be provided to the patient in any feasible order. Since one applicator (e.g., the combination applicator 900) is used to deliver multiple types of therapies, the patient may advantageously use the single applicator and associated control unit to receive different therapies, thereby simplifying the delivery of the therapies to the patent.

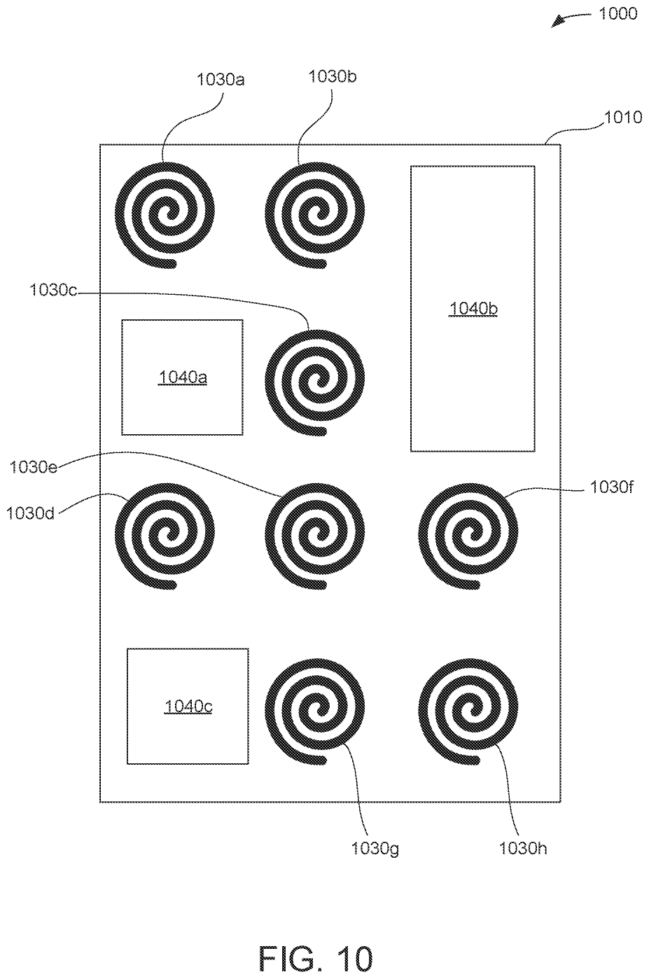

FIG. 10 shows another example combination applicator 1000, in accordance with some embodiments. The combination applicator 1000 may include a substrate 1010, PEMF emitter coils 1030a-1030h (collectively referred to as PEMF emitter coils 1030), and treatment pads 1040a-1040c (collectively referred to as treatment pads 1040). The PEMF emitter coils 1030 and the treatment pads 1040 may be similar to the PEMF emitter coils 730 and treatment pad 740 of FIG. 7, respectively.

Although shown as rectangular, in other variations the substrate 1010 may be any feasible shape. The treatment pads 1040 may all be similar or may be different from each other. Furthermore, although the combination applicator 1000 includes three treatment pads 1040, any feasible number of treatment pads may be included. In a similar manner, although eight PEMF emitter coils 1030 are shown, in other embodiments, the combination applicator 1000 may include any feasible number of PEMF emitter coils. Positions of the PEMF emitter coils 1030 and the treatment pads 1040 with respect to the substrate 1010 in FIG. 10 is meant to be illustrative rather than limiting.

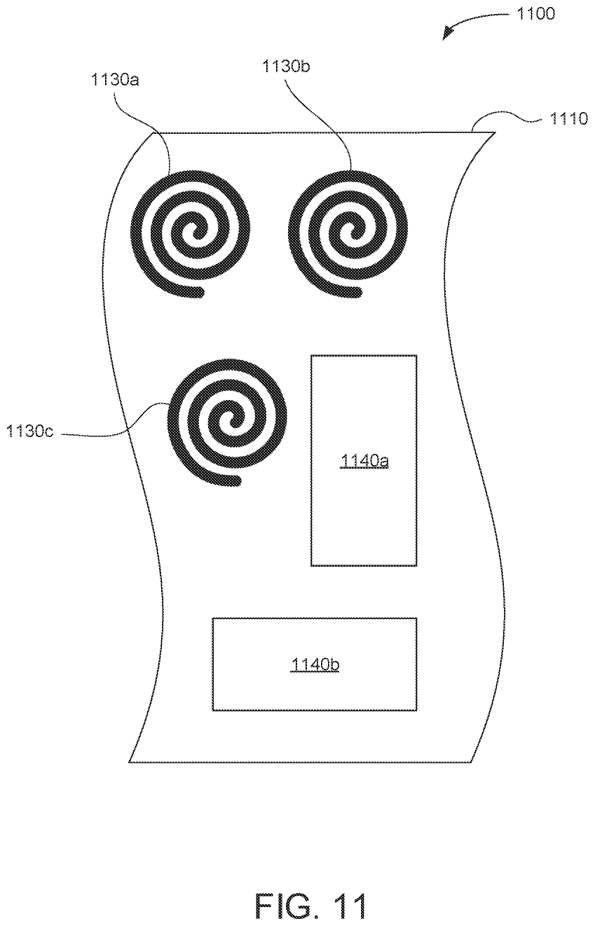

FIG. 11 shows another example combination applicator 1100, in accordance with some embodiments. The combination applicator 1100 may include a substrate 1110, PEMF emitter coils 1130a-1130c (collectively referred to as PEMF emitter coils 1130), and treatment pads 1140a-1040b (collectively referred to as treatment pads 1140). The PEMF emitter coils 1130 and the treatment pads 1140 may be similar to the PEMF emitter coils 730 and treatment pad 740 of FIG. 7, respectively. In other variations, the substrate 1110 may be any feasible shape. Positions of the PEMF emitter coils 1130 and the treatment pads 1140 with respect to the substrate 1110 in FIG. 11 is meant to be illustrative rather than limiting.

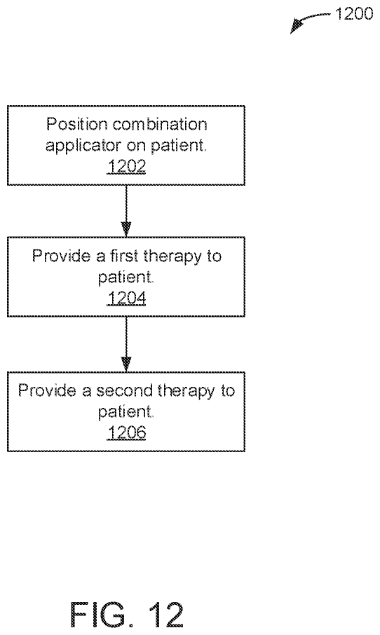

FIG. 12 is a flowchart depicting an example of one method 1200 for providing therapy to a patient with a combination applicator. Some examples may perform the operations described herein with additional operations, fewer operations, operations in a different order, operations in parallel, and some operations differently. The operations herein are described as being performed by the PEMF therapy device 110 and the PEMF applicator 120 of FIG. 1 for case of explanation. In some variations, PEMF applicator 120 may be a combination applicator, for example, as described with respect to FIGS. 7-11. Persons having skill in the art will recognize that the operations can be performed using any feasible device or system that may be configured to deliver and/or provide the therapies described herein.

In FIG. 12, the method 1200 may begin as the combination applicator is positioned on the patient 1202. In some cases, the combination applicator may be placed, positioned, or attached to a portion of the patient's body to receive at least one therapy provided by the combination applicator. In another example, the combination applicator may be configured to receive a portion of the patient's body. For example, the combination applicator may be shaped to receive a patient's arm, leg, hand, foot, finger, or the like as the patient's body part is placed in contact with the combination applicator.

Next, the PEMF therapy device 110 may provide a first therapy through the combination applicator 1204. For example, the first therapy may be a PEMF therapy. The PEMF therapy device 110 may cause PEMF emitter coils within the combination applicator to radiate a magnetic field to the patient. In another example, the first therapy may be an alternative therapy, such as a heating, cooling, or TENS therapy. Thus, the PEMF therapy device 110 may cause therapy pads within the combination applicator to deliver the alternative therapy.

Next, the PEMF therapy device 110 may provide a second therapy through the combination applicator 1206. In some variations, the second therapy can be different than the first therapy. For example, if the first therapy was a PEMF therapy, then the second therapy may be an alternative therapy. In another example, if the first therapy was an alternative therapy, then the second therapy may be a PEMF therapy.

In some variations, the first therapy may have a first time duration and the second therapy may have a second time duration. The time durations may be the same or may be different. In some other variations, the times durations may partially or completely overlap. Thus, in some variations, the patient may receive the first therapy and the second therapy concurrently.

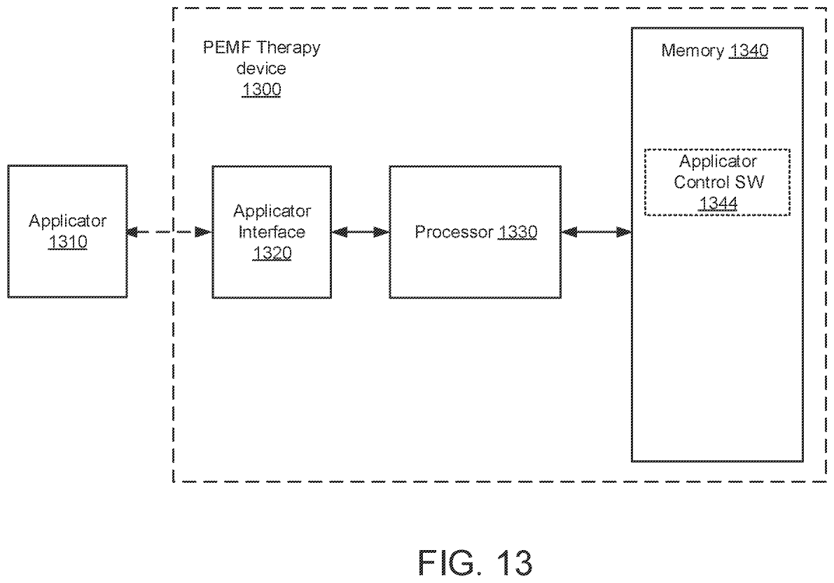

FIG. 13 shows a block diagram of a PEMF therapy device 1300 that may be one example of the PEMF therapy device 110 of FIG. 1. The PEMF therapy device 1300 may include an applicator interface 1320, a processor 1330, and a memory 1340.

The applicator interface 1320, which is also coupled to the processor 1330, may be used to interface and control any feasible PEMF applicator, such as PEMF applicator 120, applicator 1310, and/or any feasible applicator. The applicator interface 1320 may provide a high-power pulsed electromagnetic field signal to a PEMF applicator. The PEMF applicator, in return, may emit an electromagnetic field, such as a magnetic field, that may treat and penetrate body tissues. In some embodiments, the applicator interface 1320 may include driver circuitry (not shown) to generate the high-power pulsed electromagnetic field signals for the PEMF applicators. In some other embodiments, the applicator interface 1320 may also control therapy pads (for example, heating, cooling, and/or TENS pads) in the combination applicator.

The processor 1330, which is also coupled to the applicator interface 1320, and the memory 1340, may be any one or more suitable processors capable of executing scripts or instructions of one or more software programs stored in the PEMF therapy device 1300 (such as within memory 1340).

The memory 1340 may include a non-transitory computer-readable storage medium (e.g., one or more nonvolatile memory elements, such as EPROM, EEPROM, Flash memory, a hard drive, etc.) that may store an applicator control software module 1344. The applicator control software module 1344 includes program instructions that, when executed by the processor 1330, may cause the PEMF therapy device 1300 to perform the corresponding function(s). Thus, the non-transitory computer-readable storage medium of memory 1340 may include instructions for performing all or a portion of the operations described herein.

The processor 1330 may execute the applicator control software module 1344 to control operations of an applicator such as applicator 1310 coupled to the applicator interface 1320. For example, execution of the applicator control software module 1344 may cause the applicator interface 1320 to provide a high-power pulsed electromagnetic field signal to the applicator 1310, thereby enabling the applicator 1310 to provide a PEMF therapy. In another example, execution of the applicator control software module 1344 may cause the applicator interface 1320 to cause the applicator 1310 to deliver an alternative therapy such as a heating, cooling, or TENS therapy. In some variations, execution of the applicator control software module 1344 may control a time duration of the PEMF therapy and/or the alternative therapy. For example, execution of the applicator control software module 1344 may cause a PEMF therapy to be delivered for a first time period and an alternative therapy to be delivered for a second time period. In some cases, the first time period may overlap with the second time period.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached, or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising.” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components, or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims. The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A pulsed electromagnetic field (PEMF) therapy applicator comprising:
a substrate;
a ferromagnetic field director comprising a first side attached to a first surface of the substrate and a second side opposite to the first side; and
an emitter coil array disposed on the second side of the ferromagnetic field director,
wherein the ferromagnetic field director is configured to receive a magnetic field from the emitter coil array and direct the received magnetic field back toward the emitter coil array.

2. The PEMF therapy applicator of claim 1, wherein the emitter coil array includes a first PEMF emitter coil configured to emit a magnetic field toward the ferromagnetic field director and a second PEMF emitter coil configured to emit a magnetic field away from the ferromagnetic field director.

3. The PEMF therapy applicator of claim 2, wherein the first PEMF emitter coil and the second PEMF emitter coil are both configured to spiral in the same direction.

4. The PEMF therapy applicator of claim 2, wherein the first PEMF emitter coil and the second PEMF emitter coil are both configured to spiral in the opposite direction.

5. The PEMF therapy applicator of claim 2, wherein the first PEMF emitter coil is configured to emit a magnetic field having a north-south orientation toward the ferromagnetic field director and the second PEMF emitter coil is configured to emit a magnetic field having a north-south orientation away from the ferromagnetic field director.

6. The PEMF therapy applicator of claim 2, wherein the first PEMF emitter coil is disposed adjacent to the second PEMF emitter coil.

7. The PEMF therapy applicator of claim 2, wherein the second PEMF emitter coil is coupled in series with the first PEMF emitter coil.

8. The PEMF therapy applicator of claim 7, wherein current flow in the first and second PEMF emitter coils are in opposite directions with respect to a PEMF emitter coil spiral direction.

9. The PEMF therapy applicator of claim 2, wherein the ferromagnetic field director is further configured to direct the magnetic field from the first PEMF emitter coil to the second PEMF emitter coil.

10. The PEMF therapy applicator of claim 2, wherein the ferromagnetic field director includes a plurality of individual ferromagnetic field directors, wherein each individual ferromagnetic field director is disposed under two PEMF emitter coils of the emitter coil array.

11. The PEMF therapy applicator of claim 1, wherein the ferromagnetic field director includes at least one metal selected from the group consisting of cobalt, iron, nickel, and gadolinium.

12. The PEMF therapy applicator of claim 1, wherein the substrate is a printed circuit board.

13. The PEMF therapy applicator of claim 1 further comprising an adhesive configured to temporarily affix the PEMF therapy applicator to a selected body part.

14. The PEMF therapy applicator of claim 1 further comprising a visual cue configured to guide positioning of a body part with respect to the emitter coil array.

15. A pulsed electromagnetic field (PEMF) system comprising:
a PEMF therapy device, configured to control a generation of a PEMF signal; and
a PEMF applicator coupled to the PEMF therapy device and configured to deliver the PEMF signal to a patient, the PEMF applicator comprising:
a substrate;
a ferromagnetic field director comprising a first side attached to a first surface of the substrate and a second side opposite to the first side; and
an emitter coil array disposed on the second side of the ferromagnetic field director,
wherein the ferromagnetic field director is configured to receive a magnetic field from the emitter coil array and direct the received magnetic field back toward the emitter coil array.

16. The PEMF system of claim 15, wherein the emitter coil array includes a first PEMF emitter coil configured to emit a magnetic field toward the ferromagnetic field director and a second PEMF emitter coil configured to emit a magnetic field away from the ferromagnetic field director.

17. The PEMF system of claim 16, wherein the first PEMF emitter coil and the second PEMF emitter coil are both configured to spiral in the same direction.

18. The PEMF system of claim 16, wherein the first PEMF emitter coil and the second PEMF emitter coil are both configured to spiral in the opposite directions.

19. The PEMF system of claim 16, wherein the first PEMF emitter coil is configured to emit a magnetic field having a north-south orientation toward the ferromagnetic field director and the second PEMF emitter coil is configured to emit a magnetic field having a north-south orientation away from the ferromagnetic field director.

20. The PEMF system of claim 16, wherein the first PEMF emitter coil is disposed adjacent to the second PEMF emitter coil.

21. The PEMF system of claim 16, wherein the second PEMF emitter coil is coupled in series with the first PEMF emitter coil.

22. The PEMF system of claim 21, wherein current flow in the first and second PEMF emitter coils are in opposite directions with respect to a PEMF emitter coil spiral direction.

23. The PEMF system of claim 16, wherein the ferromagnetic field director is further configured to direct the magnetic field from the first PEMF emitter coil to the second PEMF emitter coil.

24. The PEMF system of claim 16, wherein the ferromagnetic field director includes a plurality of individual ferromagnetic field directors, wherein each individual ferromagnetic field director is disposed under two PEMF emitter coils of the emitter coil array.

25. The PEMF system of claim 15, wherein the ferromagnetic field director includes at least one metal selected from the group consisting of cobalt, iron, nickel, and gadolinium.

26. The PEMF system of claim 15, wherein the substrate is a printed circuit board.

27. The PEMF system of claim 15, wherein the PEMF applicator further comprises an adhesive configured to temporarily affix the PEMF applicator to a selected body part.

28. The PEMF system of claim 15, wherein the PEMF applicator further comprises a visual cue configured to guide positioning of a body part with respect to the emitter coil array.

29-39. (canceled)
